(19) United States
(12) Patent Application Publication
(10) Pub. No.: US 2006/0286120 A1
(43) Pub. Date: Dec. 21, 2006

(54) Method of drying a plant product without using monosodium glutamate, and a product obtained by implementing the method

(75) Inventor: Luc Darbonne, Oncy Sur Ecole (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Darome, a corporation of France, Milly la Foret (FR)

(21) Appl. No.: 11/454,199

(22) Filed: Jun. 16, 2006

(30) Foreign Application Priority Data
Jun. 17, 2005 (FR) 05/06175

(51) Int. Cl. A61K 36/18; A23C 1/06; A61K 36/06

(52) U.S. Cl. 424/195.16; 424/725; 426/384; 426/385

(57) ABSTRACT

A method of drying a spice or aromatic herb plant product including providing a quantity Q₀ of frozen plant product; adding a quantity qₑ of an electrolyte and mixing with a quantity q_{g} of a glucid to obtain a homogeneous quantity Q₁ of an edible water-soluble carrier substance, the carrier substance being substantially free of monosodium glutamate (MSG); adding the quantity Q₀ of frozen plant product and mixing with the quantity Q₁ of carrier substance to obtain a resulting frozen plant product with which the carrier substance has been mixed substantially homogeneously; and heating the resulting product to a temperature in the range 50° C. to 170° C. for a time sufficient to substantially dry the product; and a plant product produced by the method, the product including, by weight, less than about 3% of water, about 20% to about 50% of plant matter, and about 35% to about 65% of electrolyte.

### RELATED APPLICATION

This application claims priority of French Patent Application No. 05/06175, filed Jun. 17, 2005, herein incorporated by reference.

### TECHNICAL FIELD

The invention relates to a method of drying a plant product of the spice or aromatic herb type, and to a plant product dried using the method.

### BACKGROUND

Many recipes require the use of spices or aromatic herbs that, for reasons of logistics and industrial constraints, are preferably packaged in dried form. However, the constraints for use must not be satisfied to the detriment, in particular, of aroma, appearance (shape, color, etc.) and mechanical strength of the plant product implemented.

EP-B-0 087 717 discloses a flowable dried aromatic vegetable product that is formed of dried and comminuted aromatic herbs and/or spices. To obtain a dried product that has aroma and color that match those of the fresh product, EP-B-0 087 717 teaches that the mixture to be dried can be buffered by adding monosodium glutamate (MSG) to raise the pH of the cell liquor or sap of the product during drying.

Although such products have been marketed widely and given satisfaction, it appears desirable, in particular for reasons of consumer acceptance, to omit the use of MSG, naturally without degrading the quality of the dried product obtained.

### SUMMARY

This invention relates to a method of drying a spice or aromatic herb plant product including providing a quantity Q₁₀ of frozen plant product, adding a quantity qₑ of an electrolyte and mixing with a quantity q_{g} of a glucid to obtain a homogeneous quantity Q₁ of an edible water-soluble carrier substance, the carrier substance being substantially free of monosodium glutamate (MSG), adding the quantity Q₀ of frozen plant product and mixing with the quantity Q₁ of carrier substance to obtain a resulting frozen plant product with which the carrier substance has been mixed substantially homogeneously, and heating the resulting product to a temperature in the range of about 50° C. to about 170° C. for a time sufficient to substantially dry the product.

This invention also relates to a plant product produced by the method, the product including, by weight, less than about 3% of water, about 20% to about 50% of plant matter, and about 35% to about 65% of electrolyte.

### DETAILED DESCRIPTION

I conducted intensive tests and discovered a method making it possible to obtain a dried plant product of the spice or aromatic herb type that, in particular, has an aroma and a color that match the fresh product, without using MSG.

To this end, and in a first aspect, I discovered a method of drying a plant product of the spice or aromatic herb type comprising the following steps:
providing a quantity Q₀ of frozen plant product;
adding a quantity qₑ of an electrolyte and mixing it with a quantity q_{g} of a glucid to obtain a homogeneous quantity Q₁ of an edible water-soluble carrier substance, the carrier substance being free of monosodium glutamate (MSG);
adding the quantity Q₀ of frozen plant product and mixing it with the quantity Q₁ of carrier substance to obtain a resulting frozen plant product with which the carrier substance has been mixed homogeneously; and
heating the resulting product to a temperature in the range of about 50° C. to about 170° C. for a time sufficient to obtain drying.

In another aspect, I discovered a plant product of the spice or aromatic herb type, the product being dried by the above method, the product comprising, by weight, less than about 3% of water, in the range of about 20% to about 50% of plant matter, and in the range of about 35% to about 65% of electrolyte.

The product may be used for preparing soups, sauces and cooked dishes, as a coating product for dry foodstuffs or as a condiment.

Thus, the method relates to drying a plant product of the spice or aromatic herb type, wherein the dried product may be used in food preparations such as soups, sauces, cooked dishes and the like, or indeed as a coating product for dry foodstuffs.

By way of example, the plant product can be chosen from dill, basil, celery, chervil, coriander, watercress, spinach, estragon, fennel, lovage, marjoram, mint, sorrel, parsley, leek, rosemary, savory, thyme, sage, oregano, lavender, chives, garlic, shallot, onion, paprika, rocket and the like, and mixtures thereof.

Advantageously, this type of plant product is preserved substantially immediately after being harvested, by any known freezing method. The frozen plant product can be in the form of the whole plant or in the form of fractions of the plant (e.g. leafs, seeds, etc.).

The method makes provision to dry a quantity Q₀ of plant product that is pre-frozen, in particular at a temperature lying in the range of about −15° C. to about −20° C..

In a step of the method, a quantity qₑ of an electrolyte is added and mixed with a quantity q_{g} of a glucid to obtain a homogeneous quantity Q₁ of an edible water-soluble carrier substance. This step can be performed in a food powder mixer of conventional structure.

The electrolyte may comprise sodium chloride and/or potassium chloride, and it is also advantageous to use a hydrogen-containing glucid. Such a mixture imparts satisfactory aroma preservation to the dried plant product. However, in particular, electrolytes that are less rich in salt can be used.

The method then makes provision to add the quantity Q₀ of frozen plant product and mix it with the quantity Q₁ of carrier substance to obtain a resulting frozen plant product with which the carrier substance has been mixed homogeneously. In particular, during this step, the plant product can be maintained at a temperature lying in the range of about −15° C. to about −20° C. to keep it frozen. This step may be performed in a food mixer that is thermally insulated.

The quantity Q₀ implemented can be in the range of about 60% of the total weight to about 80% of the total weight, in particular the range of about 65% by weight to about 75% by weight. In particular, after the above-mentioned step, the frozen plant product may be covered by the carrier substance.

In the next step, the method makes provision to heat the resulting product to a temperature in the range of about 50° C. to about 170° C., for a time sufficient to obtain drying. In particular, drying is to be understood to mean bringing the residual water content in the plant product to less than about 3%. This step can be performed by disposing the resulting product in a fine layer on an endless belt to pass the product through a heater tunnel for the necessary time. The heater tunnel can have a temperature gradient to improve the drying.

I discovered that, by performing the above-mentioned steps, it is possible to use a carrier substance that is free of monosodium glutamate (MSG), while obtaining a satisfactory quality for the dried plant product. This quality is expressed in particular both from the point of view of organoleptic properties such as aroma (shown by essential oil content and by a board of tasters) and from the point of view of color properties (shown by measurements).

The resulting product may be heated to a maximum temperature in the range of about 125° C. to about 135° C..

It is also possible to make provision for the carrier substance to contain a quantity q₁ of a nucleotide-rich yeast extract, the extract being added and mixed at the same time as the electrolyte and the glucid to obtain a homogeneous carrier substance.

A yeast extract that is rich in guanosine 5′-monophosphate (GMP) and/or in inosine 5′-monophosphate (IMP) is particularly satisfactory as a taste enhancer.

In addition, the quantity q₁ of yeast extract implemented can be in the range of about 3% by weight of the quantity Q₁ of carrier substance to about 6% by weight of said quantity Q₁, which is smaller than the quantity of MSG conventionally used. Therefore, implementing the method makes it possible to obtain a dried plant product that is richer in plant matter.

I also discovered that it is advantageous for the quality of the dried product obtained, for the quantity Q₁ of carrier substance to be determined as a function of the weight of dry matter of the quantity Q₀ of frozen plant product.

Indeed, as a function of harvests, the quantity of dry matter can vary to a large extent, which, if the quantity of carrier substance remains constant, can cause quality dispersions in the dried product.

In particular, for any given dried plant product, it is possible to determine and use a constant ratio between the weight of dry matter and the weight of the quantity Q₁ of carrier substance.

I also discovered a plant product of the spice or aromatic herb type that is dried by implementing the method as described above, the product comprising, by weight, less than about 3% of water, in the range of about 20% to about 50% of plant matter, and in the range of about 35% to about 65% of electrolytes. The plant product may further comprise at least about 3% by weight of nucleotide-rich yeast extract.

Two representative examples are given below.

### EXAMPLE 1

Frozen plant product: basil; Q₀=710 g
Carrier substance: 72.4% of salt, 23.1% of glucid, 4.5% of yeast extract rich in sodium guanylate and inosinate; Q₁=290 g

Heating for two and a half hours with a temperature gradient of 130° C. to 60° C.
Dried product: 1.3% of water; 50.8% of salt; 29.8% of plant matter

The color and the aroma were tested and were satisfactory.

### EXAMPLE 2

Same conditions as in Example 1, with parsley.
Dried product: 1.2% of water; 53.2% of salt; 26.5% of plant matter

The color and the aroma were tested and were satisfactory.

1. A method of drying a spice or aromatic herb plant product comprising:
providing a quantity Q₀ of frozen plant product;
adding a quantity qₑ of an electrolyte and mixing with a quantity q_{g} of a glucid to obtain a homogeneous quantity Q₁ of an edible water-soluble carrier substance, the carrier substance being substantially free of monosodium glutamate (MSG);
adding the quantity Q₀ of frozen plant product and mixing with the quantity Q₁ of carrier substance to obtain a resulting frozen plant product with which the carrier substance has been mixed substantially homogeneously; and
heating the resulting product to a temperature in the range 50° C. to 170° C. for a time sufficient to substantially dry the product.

2. The method according to claim 1, wherein the plant product is frozen at a temperature in the range of about −15° C. to about −20° C.

3. The method according to claim 1, wherein the electrolyte comprises sodium chloride and/or potassium chloride.

4. The method according to claim 1, wherein the glucid is a hydrogen-containing glucid.

5. The method according to claim 1, wherein the resulting product is heated to a maximum temperature in the range of about 125° C. to about 135° C.

6. The method according to claim 1, wherein the carrier substance further comprises a quantity q₁ of a yeast extract rich in nucleotides.

7. The method according to claim 6, wherein the yeast extract is rich in guanosine 5′-monophosphate (GMP) and/or in inosine 5′-monophoshate (IMP).

8. The method according to claim 6, wherein the quantity q₁ of yeast extract is in the range of about 3% by weight to about 6% by weight of the quantity Q₁ of carrier substance.

9. The method according to claim 1, wherein the quantity Q₁ of carrier substance is determined as a function of the weight of dry matter of the quantity Q₀ of frozen plant product.

10. The method according to claim 9, wherein a ratio between the weight of dry matter and the weight of the quantity Q₁ of carrier substance is determined for a given plant product.

11. The method according to claim 1, wherein the quantity Q₀ is in the range of about 60% of the total weight implemented to about 80% of the total weight.

12. The method according to claim 1, wherein the quantity Q₀ is in the range of about 65% of the total weight implemented to about 75% of the total weight.

13. A plant product produced by the method according to claim 1, the product comprising, by weight, less than about 3% of water, about 20% to about 50% of plant matter, and about 35% to about 65% of electrolyte.

14. The dried plant product according to claim 13, further comprising at least about 3% by weight of a yeast extract rich in nucleotides.
